(19) United States
(12) Patent Application Publication
(10) Pub. No.: US 2026/0284340 A1
(43) Pub. Date: Sep. 24, 2026

(54) CPAP APPARATUS, CPAP CONFIGURATION DATA UPDATE SYSTEM, CPAP CONFIGURATION DATA UPDATE PROGRAM, AND CPAP CONFIGURATION DATA UPDATE METHOD

(71) Applicant: Murata Manufacturing Co., Ltd., Nagaokakyo-shi (JP)

(72) Inventors: Hiroaki MIEDA, Nagaokakyo-shi (JP); Takemasa MURATA, Nagaokakyo-shi (JP); Kenya HASEGAWA, Nagaokakyo-shi (JP)

(21) Appl. No.: 19/574,863

(22) Filed: Mar. 23, 2026

(30) Foreign Application Priority Data
Mar. 24, 2025 (JP) 2025-048390

(51) Int. Cl. A61M 16/00; G16H 40/60

(52) U.S. Cl. CPC A61M 16/022; G16H 40/60

(57) ABSTRACT

A CPAP apparatus includes a storage device that stores configuration data and an execution device that manages the configuration data. The configuration data includes a modifiable user setting and a basic setting that is not modifiable by a specific user. When an update of the configuration data including the basic setting is requested and the update time of the user setting in the storage device is on or before the generation time of the configuration data to be obtained to perform the update, the execution device updates the basic setting and the user setting in the configuration data. Conversely, when the update time of the user setting in the storage device is after the generation time of the configuration data to be obtained to perform the update, the execution device updates the basic setting in the configuration data while keeping the user setting in the configuration data unchanged.

### CROSS REFERENCE TO RELATED APPLICATION

This application claims priority from Japanese Patent Application No. 2025-048390, filed on Mar. 24, 2025. The content of these applications are incorporated herein by reference in its entirety.

### BACKGROUND OF THE DISCLOSURE

### 1. Field of the Disclosure

The present disclosure relates to a CPAP apparatus, a CPAP configuration data update system, a CPAP configuration data update program, and a CPAP configuration data update method.

### 2. Description of the Related Art

A CPAP apparatus disclosed in International Publication No. 2024/116638 includes a mask, a hose, a flow generator, and a control device. The mask is worn to cover the nose or mouth of a user of the CPAP apparatus, that is, a patient using the CPAP apparatus. The flow generator is connected to the mask via the hose. The flow generator is capable of delivering intake air under pressure to the mask via the hose. The control device controls the operation of the flow generator by outputting control signals to the flow generator. Here, “CPAP” is an abbreviation for “continuous positive airway pressure”.

### BRIEF SUMMARY OF THE DISCLOSURE

A control device of a CPAP apparatus as described in International Publication No. 2024/116638 generally stores configuration data related to the operation of the CPAP apparatus. For example, the configuration data of the CPAP apparatus may be updated by the user by operating operation buttons of the CPAP apparatus. As another example, the configuration data may be updated via a communication line or a portable storage medium. When the CPAP apparatus is capable of receiving configuration data from an external source, configuration data updated by the user may be updated by the configuration data received from the external source without the user's intent, which may confuse the user.

To solve the above problem, a CPAP apparatus includes a storage device that stores configuration data of the CPAP apparatus and an execution device. The configuration data includes a modifiable user setting and a basic setting that is not modifiable by a specific user. The execution device performs a user update process for updating the user setting in the configuration data in response to a request by the specific user to update the user setting; a storage process for storing an update time of the user setting as update time data in the storage device when the user setting is updated by the user update process; a generation time acquisition process for obtaining, when an update of the configuration data including the basic setting is requested, generation time data indicating a generation time of the configuration data to be obtained to perform the update; a first update process for updating the basic setting and the user setting in the configuration data stored in the storage device with the basic setting and the user setting in the configuration data associated with the generation time data when the update time indicated by the update time data stored in the storage device is on or before the generation time indicated by the generation time data; and a second update process for updating the basic setting in the configuration data stored in the storage device with the basic setting in the configuration data associated with the generation time data while keeping the user setting in the configuration data stored in the storage device unchanged when the update time indicated by the update time data stored in the storage device is after the generation time indicated by the generation time data.

To solve the above problem, a CPAP configuration data update system includes a CPAP apparatus including a client-side storage device that stores configuration data of the CPAP apparatus and a client-side execution device; and a server including a server-side storage device that stores the configuration data and a server-side execution device that updates the configuration data of the CPAP apparatus. The configuration data includes a modifiable user setting and a basic setting that is not modifiable by a specific user. The client-side execution device performs a user update process for updating the user setting in the configuration data in response to a request by the specific user to update the user setting and a storage process for storing an update time of the user setting as update time data in the client-side storage device when the user setting is updated by the user update process. The server-side execution device performs a generation time acquisition process for obtaining generation time data indicating a generation time of the configuration data stored in the server-side storage device when an update of the configuration data including the basic setting at the CPAP apparatus is requested; an update time acquisition process for obtaining the update time data stored in the client-side storage device when an update of the configuration data including the basic setting at the CPAP apparatus is requested; a first update process for updating the basic setting and the user setting in the configuration data stored in the client-side storage device with the basic setting and the user setting in the configuration data associated with the generation time data by transmitting the basic setting and the user setting in the configuration data associated with the generation time data to the CPAP apparatus when the update time indicated by the update time data is on or before the generation time indicated by the generation time data; and a second update process for updating the basic setting in the configuration data stored in the client-side storage device with the basic setting in the configuration data associated with the generation time data while keeping the user setting in the configuration data stored in the client-side storage device unchanged by transmitting the basic setting in the configuration data associated with the generation time data to the CPAP apparatus when the update time indicated by the update time data is after the generation time indicated by the generation time data.

To solve the above problem, a CPAP configuration data update program is provided for a CPAP apparatus including a storage device that stores configuration data of the CPAP apparatus and an execution device. The configuration data includes a modifiable user setting and a basic setting that is not modifiable by a specific user. The CPAP configuration data update program causes the execution device to perform a user update process for updating the user setting in the configuration data in response to a request by the specific user to update the user setting; a storage process for storing an update time of the user setting as update time data in the storage device when the user setting is updated by the user update process; a generation time acquisition process for obtaining, when an update of the configuration data including the basic setting is requested, generation time data indicating a generation time of the configuration data to be obtained to perform the update; a first update process for updating the basic setting and the user setting in the configuration data stored in the storage device with the basic setting and the user setting in the configuration data associated with the generation time data when the update time indicated by the update time data stored in the storage device is on or before the generation time indicated by the generation time data; and a second update process for updating the basic setting in the configuration data stored in the storage device with the basic setting in the configuration data associated with the generation time data while keeping the user setting in the configuration data stored in the storage device unchanged when the update time indicated by the update time data stored in the storage device is after the generation time indicated by the generation time data.

To solve the above problem, a CPAP configuration data update method is provided for a CPAP apparatus including a storage device that stores configuration data of the CPAP apparatus and an execution device. The configuration data includes a modifiable user setting and a basic setting that is not modifiable by a specific user. In the CPAP configuration data update method, the execution device performs a user update process for updating the user setting in the configuration data in response to a request by the specific user to update the user setting; a storage process for storing an update time of the user setting as update time data in the storage device when the user setting is updated by the user update process; a generation time acquisition process for obtaining, when an update of the configuration data including the basic setting is requested, generation time data indicating a generation time of the configuration data to be obtained to perform the update; a first update process for updating the basic setting and the user setting in the configuration data stored in the storage device with the basic setting and the user setting in the configuration data associated with the generation time data when the update time indicated by the update time data stored in the storage device is on or before the generation time indicated by the generation time data; and a second update process for updating the basic setting in the configuration data stored in the storage device with the basic setting in the configuration data associated with the generation time data while keeping the user setting in the configuration data stored in the storage device unchanged when the update time indicated by the update time data stored in the storage device is after the generation time indicated by the generation time data.

To solve the above problem, a CPAP configuration data update program is provided for an update system that includes a CPAP apparatus including a client-side storage device that stores configuration data of the CPAP apparatus and a client-side execution device; and a server including a server-side storage device that stores the configuration data and a server-side execution device that updates the configuration data of the CPAP apparatus. The configuration data includes a modifiable user setting and a basic setting that is not modifiable by a specific user. The CPAP configuration data update program causes the client-side execution device to perform a user update process for updating the user setting in the configuration data in response to a request by the specific user to update the user setting and a storage process for storing an update time of the user setting as update time data in the client-side storage device when the user setting is updated by the user update process, and causes the server-side execution device to perform a generation time acquisition process for obtaining generation time data indicating a generation time of the configuration data stored in the server-side storage device when an update of the configuration data including the basic setting at the CPAP apparatus is requested, an update time acquisition process for obtaining the update time data stored in the client-side storage device when an update of the configuration data including the basic setting at the CPAP apparatus is requested, a first update process for updating the basic setting and the user setting in the configuration data stored in the client-side storage device with the basic setting and the user setting in the configuration data associated with the generation time data by transmitting the basic setting and the user setting in the configuration data associated with the generation time data to the CPAP apparatus when the update time indicated by the update time data is on or before the generation time indicated by the generation time data, and a second update process for updating the basic setting in the configuration data stored in the client-side storage device with the basic setting in the configuration data associated with the generation time data while keeping the user setting in the configuration data stored in the client-side storage device unchanged by transmitting the basic setting in the configuration data associated with the generation time data to the CPAP apparatus when the update time indicated by the update time data is after the generation time indicated by the generation time data.

To solve the above problem, a CPAP configuration data update method is provided for an update system that includes a CPAP apparatus including a client-side storage device that stores configuration data of the CPAP apparatus and a client-side execution device, and a server including a server-side storage device that stores the configuration data and a server-side execution device that updates the configuration data of the CPAP apparatus. The configuration data includes a modifiable user setting and a basic setting that is not modifiable by a specific user. In the CPAP configuration data update method, the client-side execution device performs a user update process for updating the user setting in the configuration data in response to a request by the specific user to update the user setting and a storage process for storing an update time of the user setting as update time data in the client-side storage device when the user setting is updated by the user update process; and the server-side execution device performs a generation time acquisition process for obtaining generation time data indicating a generation time of the configuration data stored in the server-side storage device when an update of the configuration data including the basic setting at the CPAP apparatus is requested, an update time acquisition process for obtaining the update time data stored in the client-side storage device when an update of the configuration data including the basic setting at the CPAP apparatus is requested, a first update process for updating the basic setting and the user setting in the configuration data stored in the client-side storage device with the basic setting and the user setting in the configuration data associated with the generation time data by transmitting the basic setting and the user setting in the configuration data associated with the generation time data to the CPAP apparatus when the update time indicated by the update time data is on or before the generation time indicated by the generation time data, and a second update process for updating the basic setting in the configuration data stored in the client-side storage device with the basic setting in the configuration data associated with the generation time data while keeping the user setting in the configuration data stored in the client-side storage device unchanged by transmitting the basic setting in the configuration data associated with the generation time data to the CPAP apparatus when the update time indicated by the update time data is after the generation time indicated by the generation time data.

The above configurations make it possible to prevent user settings set by the user from being updated without the user's intent when an update of configuration data including basic settings is requested.

### BRIEF DESCRIPTION OF THE SEVERAL VIEWS OF THE DRAWINGS

FIG. 1 is a schematic diagram of an update system according to a first embodiment;

FIG. 2 is a flowchart illustrating user setting update control according to the first embodiment;

FIG. 3 is a sequence diagram illustrating basic setting update control according to the first embodiment;

FIG. 4 is a sequence diagram illustrating the basic setting update control according to the first embodiment;

FIG. 5 is a sequence diagram illustrating the basic setting update control according to the first embodiment;

FIG. 6 is a sequence diagram illustrating basic setting update control according to a second embodiment;

FIG. 7 is a sequence diagram illustrating the basic setting update control according to the second embodiment; and

FIG. 8 is a sequence diagram illustrating the basic setting update control according to the second embodiment.

### DETAILED DESCRIPTION OF THE DISCLOSURE

### First Embodiment

### (Schematic Configuration of Update System)

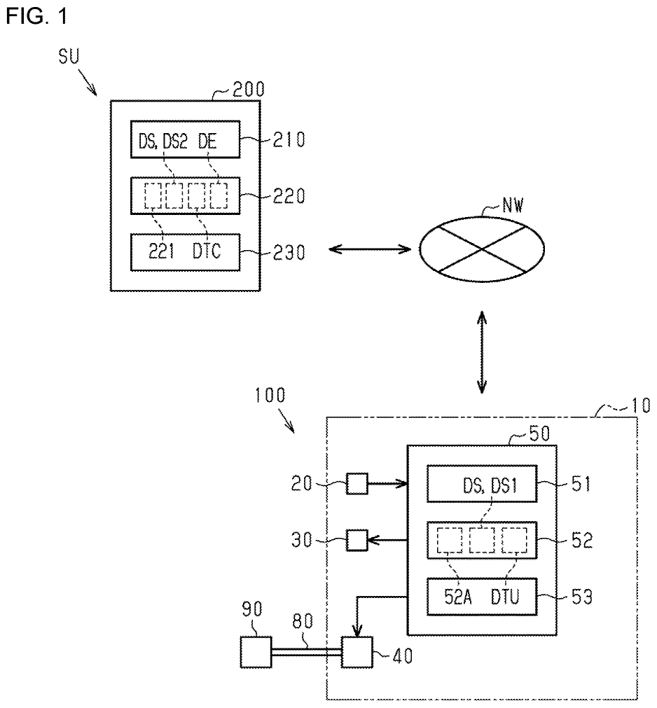

A first embodiment of the present disclosure is described below with reference to FIGS. 1 to 5. First, a schematic configuration of an update system SU is described.

As illustrated in FIG. 1, the update system SU includes multiple CPAP apparatuses 100. In the present embodiment, each CPAP apparatus 100 is used, for example, in a situation as described below. First, assume that a user of the CPAP apparatus 100, i.e., a patient who uses the CPAP apparatus 100, visits a medical institution, and a doctor of the medical institution determines that the patient requires therapy using the CPAP apparatus 100. In this case, the patient uses the CPAP apparatus 100 by renting the CPAP apparatus 100 from the medical institution. In the present embodiment, the patient using the CPAP apparatus 100 is an example of a specific user of the CPAP apparatus 100. Here, only one CPAP apparatus 100 is illustrated in FIG. 1 as a representative example.

The CPAP apparatus 100 includes a body 10, a hose 80, and a mask 90. The body 10 includes operation switches 20, a display 30, a flow generator 40, and a control device 50. The operation switches 20 are used by the user of the CPAP apparatus 100 to operate the CPAP apparatus 100. The display 30 can display various types of information.

The flow generator 40 includes a blower fan and an electric motor (not shown). A first end of the hose 80 is connected to the flow generator 40. A second end of the hose 80 is connected to the mask 90. When the CPAP apparatus 100 is used, the mask 90 is attached to the user to cover the nose or mouth of the user of the CPAP apparatus 100. The flow generator 40 delivers intake air under pressure to the mask 90 via the hose 80. The CPAP apparatus 100 is configured such that another mask 90 can be used instead of the mask 90 originally attached to the body 10. In other words, the CPAP apparatus 100 is configured such that various types of masks 90 can be used according to, for example, the size of the nose or mouth of the user of the CPAP apparatus 100.

As illustrated in FIG. 1, the control device 50 includes an execution device 51, a storage device 52, and a communication device 53. The communication device 53 can communicate with devices outside the CPAP apparatus 100 via a communication network NW. An example of the execution device 51 is a CPU. The storage device 52 includes a ROM permitting only reading, a volatile RAM permitting both reading and writing, and a non-volatile storage permitting both reading and writing. Various programs and various types of data are stored in the storage device 52 in advance. Specifically, a control program 52A is stored in the storage device 52 in advance as one of various programs. The execution device 51 executes the control program 52A to perform various processes described below. In the present embodiment, the control program 52A is an example of a CPAP configuration data update program. The execution device 51 also performs various processes related to a CPAP configuration data update method by executing the control program 52A.

Configuration data DS is also stored in the storage device 52 in advance as one of various types of data. The configuration data DS is used to set the operation of the CPAP apparatus 100. The configuration data DS includes user settings DSU and basic settings DSB. The user settings DSU can be modified by the user of the CPAP apparatus 100. For example, the user settings DSU include a setting on the type of the mask 90 and a setting on the sleep timer of the display 30. Thus, the user settings DSU are settings that have relatively small influence on the therapy using the CPAP apparatus 100. The basic settings DSB cannot be modified by the user of the CPAP apparatus 100. For example, the basic settings DSB include a setting related to the maximum pressure of air supplied from the flow generator 40 to the mask 90 and the minimum pressure of air supplied from the flow generator 40 to the mask 90. Thus, the basic settings DSB include settings that have comparatively large influence on the therapy using the CPAP apparatus 100. The execution device 51 manages the configuration data DS through various processes described below.

The storage device 52 stores update date-and-time data DTU as one of various types of data. The update date-and-time data DTU indicates the update date and time when the user settings DSU are updated. In the present embodiment, the configuration data DS and the update date-and-time data DTU are reset to initial data when the user rents the CPAP apparatus 100 from the medical institution. The initial data is, for example, data stored in the storage device 52 at the manufacturing stage of the CPAP apparatus 100. Therefore, the update date and time indicated by the update date-and-time data DTU when the user rents the CPAP apparatus 100 from the medical institution is the same as the date and time when the CPAP apparatus 100 is manufactured. For example, the update date and time indicated by the update date-and-time data DTU when the user rents the CPAP apparatus 100 from the medical institution is expressed as “15:00 on 1 Mar. 20XX”. In the present embodiment, the update date-and-time data DTU is an example of update time data indicating the update time of the user settings DSU.

The execution device 51 of the control device 50 controls the operation of the flow generator 40 by outputting control signals to the flow generator 40. The execution device 51 also outputs control signals to the display 30 to cause the display 30 to display various types of information. The control device 50 can receive operations from the user of the CPAP apparatus 100 via the operation switches 20.

As illustrated in FIG. 1, the update system SU includes a server 200. The server 200 includes an execution device 210, a storage device 220, and a communication device 230. The communication device 230 can communicate with devices outside the server 200 via the communication network NW. Specifically, the communication device 230 can communicate with the CPAP apparatus 100 via the communication network NW. An example of the execution device 210 is a CPU. The storage device 220 includes a ROM permitting only reading, a volatile RAM permitting both reading and writing, and a non-volatile storage permitting both reading and writing. Various programs and various types of data are stored in the storage device 220 in advance. Specifically, a control program 221 is stored in the storage device 220 in advance as one of various programs. The execution device 210 executes the control program 221 to perform various processes described below.

Also, the storage device 220 can store the configuration data DS as one of various types of data. In the descriptions below, configuration data DS stored in the storage device 52 of the CPAP apparatus 100 is referred to as client-side configuration data DS1, and configuration data DS stored in the storage device 220 of the server 200 is referred to as server-side configuration data DS2.

As with the client-side configuration data DS1 described above, the server-side configuration data DS2 includes user settings DSU and basic settings DSB. In the present embodiment, for example, the server-side configuration data DS2 is generated in a situation as described below. As described above, the user of the CPAP apparatus 100 visits a medical institution before using the CPAP apparatus 100. After the visit, a personnel of the medical institution prepares the server-side configuration data DS2 in accordance with a diagnosis made for the user of the CPAP apparatus 100. Accordingly, the user settings DSU and the basic settings DSB in the server-side configuration data DS2 may differ from the user settings DSU and the basic settings DSB in the client-side configuration data DS1.

The storage device 220 can also store generation date-and-time data DTC and expiration date-and-time data DE as various types of data. The generation date-and-time data DTC indicates the date and time when the server-side configuration data DS2 is generated. That is, in the present embodiment, the generation date-and-time data DTC indicates the date and time when the personnel of the medical institution generates the server-side configuration data DS2. For example, the generation date and time indicated by the generation date-and-time data DTC is expressed as “15:00 on 1 Apr. 20XX”. In the present embodiment, the generation date-and-time data DTC is an example of generation time data indicating the time when the server-side configuration data DS2 is generated.

The expiration date-and-time data DE indicates the expiration date and time of the server-side configuration data DS2. In the present embodiment, the expiration date and time indicated by the expiration date-and-time data DE is, for example, approximately several to nineteens months after the generation date and time indicated by the generation date-and-time data DTC. Accordingly, for example, the expiration date and time indicated by the expiration date-and-time data DE is expressed as “15.00 on 1 Oct. 20XX”.

### (User Setting Update Control)

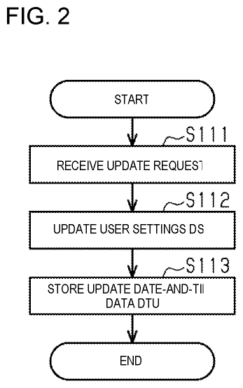

Next, user setting update control performed by the CPAP apparatus 100 is described with reference to FIG. 2. The user setting update control is performed to update the user settings DSU in the client-side configuration data DS1 in response to a change request from the user of the CPAP apparatus 100. In the present embodiment, the execution device 51 of the control device 50 of the CPAP apparatus 100 starts the user setting update control when the user of the CPAP apparatus 100 operates the operation switches 20 to update the user settings DSU.

As illustrated in FIG. 2, the execution device 51 of the control device 50 performs step S111 when the user setting update control is started. At step S111, the execution device 51 receives a request to update the user settings DSU from the user of the CPAP apparatus 100 via the operation switches 20. After step S111, the execution device 51 proceeds to step S112.

At step S112, the execution device 51 updates the user settings DSU in the client-side configuration data DS1 according to the request received at step S111. In the present embodiment, step S112 is a user update process for updating the user settings DSU in the client-side configuration data DS1 according to a request to update the user settings DSU from the user of the CPAP apparatus 100. After step S112, the execution device 51 proceeds to step S113.

At step S113, the execution device 51 stores, in the storage device 52, the date and time of the update of the user settings DSU performed at step S112 as the update date-and-time data DTU. In other words, the update date and time indicated by the update date-and-time data DTU is updated by step S113. For example, the update date and time indicated by the update date-and-time data DTU after step S113 is expressed as “15:00 on 3 Apr. 20XX”. In the present embodiment, step S113 is an example of a storage process performed to store the update date and time of the user settings DSU as the update date-and-time data DTU in the storage device 52 when the user settings DSU are updated in the user update process. After step S113, the execution device 51 terminates the current user setting update control.

### (Basic Setting Update Control)

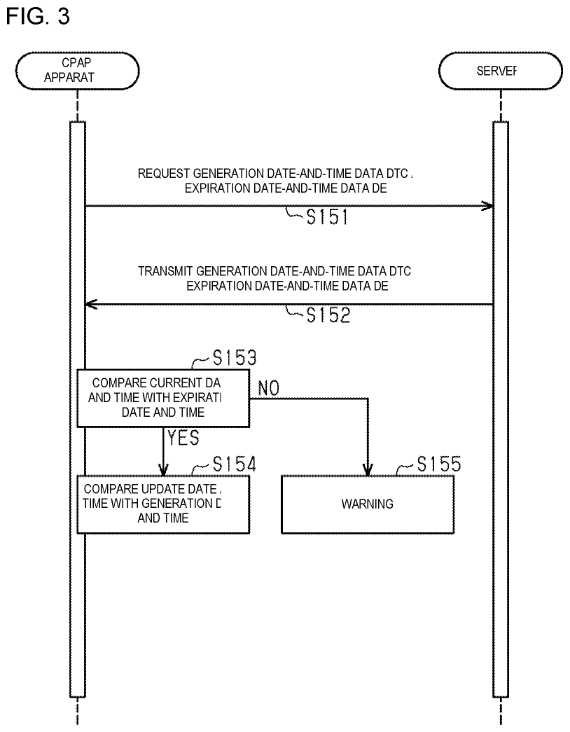

Next, the basic setting update control performed by the CPAP apparatus 100 and the server 200 is described with reference to FIGS. 3 to 5. The basic setting update control is performed to update the client-side configuration data DS1 including the basic settings DSB. The basic setting update control can be performed concurrently between multiple CPAP apparatuses 100 and the server 200. In the present embodiment, the execution device 51 of the control device 50 of the CPAP apparatus 100 starts the basic setting update control when the user of the CPAP apparatus 100 operates the operation switches 20 to update the client-side configuration data DS1 including the basic settings DSB. In other words, the execution device 51 of the control device 50 starts the basic setting update control when an update of the client-side configuration data DS1 including the basic settings DSB is requested.

As illustrated in FIG. 3, the execution device 51 of the control device 50 of the CPAP apparatus 100 performs step S151 after starting the basic setting update control. At step S151, the execution device 51 of the CPAP apparatus 100 transmits, to the server 200, a request signal for requesting the generation date-and-time data DTC and the expiration date-and-time data DE. The execution device 51 of the CPAP apparatus 100 also transmits identification information for identifying the CPAP apparatus 100 to the server 200. When the server 200 receives the request signal and so on, the execution device 210 of the server 200 proceeds to step S152.

At step S152, the execution device 210 of the server 200 transmits the generation date-and-time data DTC and the expiration date-and-time data DE to the CPAP apparatus 100. At this step, the execution device 210 of the server 200 transmits the generation date-and-time data DTC and so on for the CPAP apparatus 100 associated with the identification information obtained at step S151 to the CPAP apparatus 100 associated with the identification information obtained at step S151. When the CPAP apparatus 100 receives the generation date-and-time data DTC and the expiration date-and-time data DE, the execution device 51 of the CPAP apparatus 100 proceeds to step S153. In the present embodiment, step S152 is performed when an update of the client-side configuration data DS1 including the basic settings DSB is requested, to obtain the generation date-and-time data DTC indicating the generation date and time of the server-side configuration data DS2 to be obtained to perform the update. That is, step S152 is an example of a generation time acquisition process. Also, step S152 is an example of an expiration date-and-time acquisition process that is performed when an update of the client-side configuration data DS1 including the basic settings DSB is requested, to obtain the expiration date-and-time data DE indicating the expiration date and time of the server-side configuration data DS2 to be obtained to perform the update.

At step S153, the execution device 51 of the CPAP apparatus 100 determines whether the current date and time is on or before the expiration date and time indicated by the expiration date-and-time data DE obtained at step S152. Here, the current date and time is the date and time when step S153 is performed. In other words, the current date and time is the date and time when the update of the client-side configuration data DS1 including the basic settings DSB is requested.

When the execution device 51 determines at step S153 that the current date and time is after the expiration date and time indicated by the expiration date-and-time data DE obtained at step S152 (S153: NO), the execution device 51 proceeds to step S155.

At step S155, the execution device 51 of the CPAP apparatus 100 performs a warning process for warning the user of the CPAP apparatus 100. For example, the execution device 51 outputs a control signal to the display 30 to warn the user of the CPAP apparatus 100, via the display 30, that the current date and time is past the expiration date and time of the server-side configuration data DS2. In this case, the execution device 51 terminates the current basic setting update control.

On the other hand, when the execution device 51 determines at step S153 that the current date and time is on or before the expiration date and time indicated by the expiration date-and-time data DE obtained at step S152 (S153: YES), the execution device 51 proceeds to step S154. In other words, the execution device 51 performs one of a first update process and a second update process described below on condition that the current date and time is on or before the expiration date and time indicated by the expiration date-and-time data DE obtained at step S152.

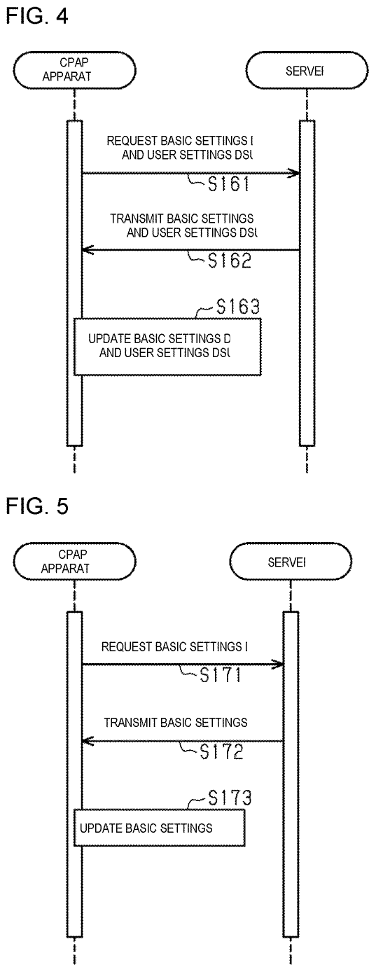

At step S154, the execution device 51 of the CPAP apparatus 100 determines whether the update date and time indicated by the update date-and-time data DTU stored in the storage device 52 is after the generation date and time indicated by the generation date-and-time data DTC obtained at step S152. When the execution device 51 determines at step S154 that the update date and time indicated by the update date-and-time data DTU is on or before the generation date and time indicated by the generation date-and-time data DTC, the execution device 51 proceeds to step S161 in FIG. 4. In other words, the execution device 51 proceeds to step S161 when the update date and time indicated by the update date-and-time data DTU is on or before the generation date and time indicated by the generation date-and-time data DTC.

As illustrated in FIG. 4, at step S161, the execution device 51 of the CPAP apparatus 100 transmits a request signal to the server 200 to request the basic settings DSB and the user settings DSU in the server-side configuration data DS2 associated with the generation date-and-time data DTC obtained at step S152. The execution device 51 of the CPAP apparatus 100 also transmits the identification information of the CPAP apparatus 100 to the server 200. When the server 200 receives the request signal and so on, the execution device 210 of the server 200 proceeds to step S162.

At step S162, the execution device 210 of the server 200 transmits the basic settings DSB and the user settings DSU in the server-side configuration data DS2 associated with the generation date-and-time data DTC to the CPAP apparatus 100. At this step, the execution device 210 of the server 200 transmits the basic settings DSB and so on of the CPAP apparatus 100 associated with the identification information obtained at step S161 to the CPAP apparatus 100 associated with the identification information obtained at step S161. When the CPAP apparatus 100 receives the server-side configuration data DS2, the execution device 51 of the CPAP apparatus 100 proceeds to step S163.

At step S163, the execution device 51 of the CPAP apparatus 100 updates the basic settings DSB and the user settings DSU in the client-side configuration data DS1 with the basic settings DSB and the user settings DSU in the server-side configuration data DS2 associated with the generation date-and-time data DTC. In the present embodiment, step S163 is an example of a first update process. After step S163, the execution device 51 terminates the current basic setting update control.

On the other hand, when the execution device 51 determines at step S154 that the update date and time indicated by the update date-and-time data DTU is after the generation date and time indicated by the generation date-and-time data DTC, the execution device 51 proceeds to step S171 in FIG. 5. In other words, the execution device 51 proceeds to step S171 when the update date and time indicated by the update date-and-time data DTU is after the generation date and time indicated by the generation date-and-time data DTC.

As illustrated in FIG. 5, at step S171, the execution device 51 of the CPAP apparatus 100 transmits a request signal to the server 200 to request the basic settings DSB in the server-side configuration data DS2 associated with the generation date-and-time data DTC obtained at step S152. The execution device 51 of the CPAP apparatus 100 also transmits the identification information of the CPAP apparatus 100 to the server 200. When the server 200 receives the request signal and so on, the execution device 210 of the server 200 proceeds to step S172.

At step S172, the execution device 210 of the server 200 transmits the basic settings DSB in the server-side configuration data DS2 associated with the generation date-and-time data DTC to the CPAP apparatus 100. At this step, the execution device 210 of the server 200 transmits the basic settings DSB of the CPAP apparatus 100 associated with the identification information obtained at step S171 to the CPAP apparatus 100 associated with the identification information obtained at step S171. When the CPAP apparatus 100 receives the server-side configuration data DS2, the execution device 51 of the CPAP apparatus 100 proceeds to step S173.

At step S173, the execution device 51 of the CPAP apparatus 100 updates the basic settings DSB in the client-side configuration data DS1 with the basic settings DSB in the server-side configuration data DS2 associated with the generation date-and-time data DTC. In this case, the execution device 51 of the CPAP apparatus 100 keeps the user settings DSU in the client-side configuration data DS1 unchanged. In the present embodiment, step S173 is an example of a second update process. After step S173, the execution device 51 terminates the current basic setting update control.

### (Effects of Present Embodiment)

As illustrated in FIGS. 3 to 5, the CPAP apparatus 100 and the server 200 perform the basic setting update control when the user of the CPAP apparatus 100 operates the operation switches 20 to update the client-side configuration data DS1 including the basic settings DSB.

As illustrated in FIG. 3, at steps S151 and S152, when an update of the client-side configuration data DS1 including the basic settings DSB is requested, the execution device 51 of the CPAP apparatus 100 obtains the generation date-and-time data DTC indicating the generation date and time of the server-side configuration data DS2 to be obtained to perform the update. At step S154, the execution device 51 of the CPAP apparatus 100 determines whether the update date and time indicated by the update date-and-time data DTU stored in the storage device 52 is after the generation date and time indicated by the generation date-and-time data DTC.

Here, the generation date and time indicated by the generation date-and-time data DTC is the date and time when the server-side configuration data DS2 is generated by a personnel of the medical institution visited by the user of the CPAP apparatus 100. The update date-and-time data DTU indicates the update date and time of the user settings DSU. In the present embodiment, the update date and time indicated by the update date-and-time data DTU when the user rents the CPAP apparatus 100 from the medical institution is the same as the date and time at which the CPAP apparatus 100 is manufactured. Therefore, for example, when the user of the CPAP apparatus 100 has not updated the user settings DSU by the user setting update control after visiting the medical institution, the update date and time indicated by the update date-and-time data DTU is on or before the generation date and time indicated by the generation date-and-time data DTC. In this case, the execution device 51 performs steps S161 to S163 after step S154. As illustrated in FIG. 4, at steps S161 and S162, the execution device 51 of the CPAP apparatus 100 obtains, from the server 200, the basic settings DSB and the user settings DSU in the server-side configuration data DS2 associated with the generation date-and-time data DTC. Then, at step S163, the execution device 51 of the CPAP apparatus 100 updates the basic settings DSB and the user settings DSU in the client-side configuration data DS1 with the basic settings DSB and the user settings DSU in the server-side configuration data DS2 associated with the generation date-and-time data DTC.

On the other hand, when, for example, the user of the CPAP apparatus 100 has updated the user settings DSU by the user setting update control after visiting the medical institution, the update date and time indicated by the update date-and-time data DTU is after the generation date and time indicated by the generation date-and-time data DTC. In this case, the execution device 51 performs steps S171 to S173 after step S154. As illustrated in FIG. 5, at steps S171 and S172, the execution device 51 of the CPAP apparatus 100 obtains, from the server 200, the basic settings DSB in the server-side configuration data DS2 associated with the generation date-and-time data DTC. Then, at step S173, the execution device 51 of the CPAP apparatus 100 updates the basic settings DSB in the client-side configuration data DS1 with the basic settings DSB in the server-side configuration data DS2 associated with the generation date-and-time data DTC while keeping the user settings DSU in the client-side configuration data DS1 unchanged.

### (Effects of Present Embodiment)

(1-1) According to the present embodiment, when an update of the client-side configuration data DS1 including the basic settings DSB is requested and the date and time of the update of the user settings DSU performed by the user of the CPAP apparatus 100 is after the generation date and time of the server-side configuration data DS2, the basic settings DSB in the client-side configuration data DS1 are updated, but the user settings DSU in the client-side configuration data DS1 are not updated. This makes it possible to prevent the user settings DSU set by the user of the CPAP apparatus 100 from being updated without the intention of the user when an update of the client-side configuration data DS1 including the basic settings DSB is requested.
(1-2) As illustrated in FIG. 3, at steps S151 and S152, when an update of the client-side configuration data DS1 including the basic settings DSB is requested, the execution device 51 of the CPAP apparatus 100 obtains the expiration date-and-time data DE indicating the expiration date and time of the server-side configuration data DS2 to be obtained to perform the update. Then, at step S153, the execution device 51 performs either steps S161 to S163 or steps S171 to S173 on condition that the current date and time is on or before the expiration date and time indicated by the expiration date-and-time data DE. That is, the execution device 51 performs one of the first update process and the second update process on condition that the current date and time is on or before the expiration date and time indicated by the expiration date-and-time data DE. With the above configuration, when an update of the client-side configuration data DS1 including the basic settings DSB is requested, it is possible to prevent the first update process and the second update process from being performed to update the client-side configuration data DS1 with the server-side configuration data DS2 that is past the expiration date and time.
(1-3) At step S153, the execution device 51 of the CPAP apparatus 100 performs a warning process to warn the user of the CPAP apparatus 100 when it is determined that the current date and time is after the expiration date and time indicated by the expiration date-and-time data DE obtained at step S152. For example, the execution device 51 outputs a control signal to the display 30 to warn the user of the CPAP apparatus 100, via the display 30, that the current date and time is past the expiration date and time of the server-side configuration data DS2. The above configuration enables the user of the CPAP apparatus 100 to know that the server-side configuration data DS2 has expired. When the user can understand the situation as described above, the user can determine that it is necessary to visit the medical institution again.

### Second Embodiment

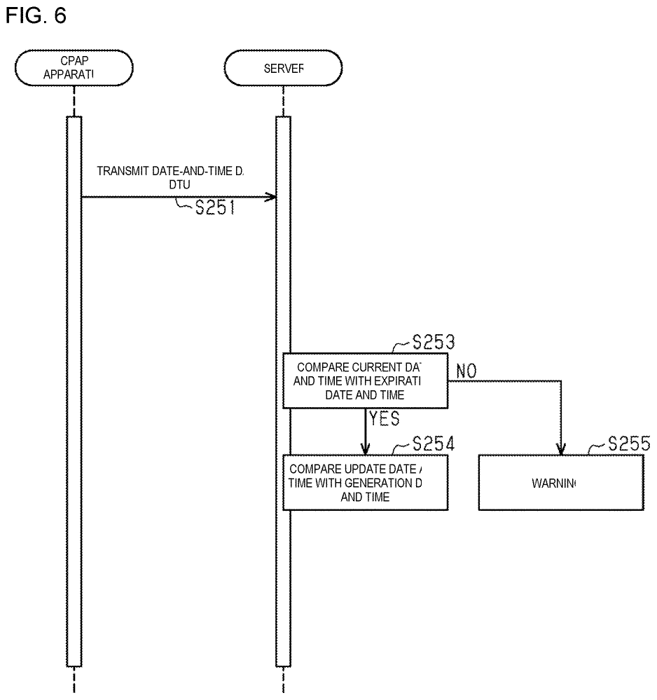

A second embodiment of the present disclosure is described below with reference to FIGS. 6 to 8. The second embodiment differs from the first embodiment in the basic setting update control. In the second embodiment, the execution device 51 of the CPAP apparatus 100 corresponds to a client-side execution device. The storage device 52 of the CPAP apparatus 100 corresponds to a client-side storage device. The execution device 210 of the server 200 corresponds to a server-side execution device. The storage device 220 of the server 200 corresponds to a server-side storage device. The control program 221 stored in the storage device 220 of the server 200 is an example of a CPAP configuration data update program. The execution device 210 of the server 200 performs various processes related to a CPAP configuration data update method by executing the control program 221. The differences of the second embodiment from the first embodiment are mainly described below. The same reference numbers as those used in the first embodiment are assigned to components corresponding to the components in the first embodiment, and descriptions of those components are omitted or simplified.

### (Basic Setting Update Control)

Next, the basic setting update control performed by the CPAP apparatus 100 and the server 200 is described with reference to FIGS. 6 to 8. The basic setting update control is performed to update the client-side configuration data DS1 including the basic settings DSB. The basic setting update control can be performed concurrently between multiple CPAP apparatuses 100 and the server 200. In the present embodiment, the execution device 51 of the control device 50 of the CPAP apparatus 100 starts the basic setting update control when the user of the CPAP apparatus 100 operates the operation switches 20 to update the client-side configuration data DS1 including the basic settings DSB. In other words, the execution device 51 of the control device 50 starts the basic setting update control when an update of the client-side configuration data DS1 including the basic settings DSB is requested.

As illustrated in FIG. 6, after starting the basic setting update control, the execution device 51 of the control device 50 of the CPAP apparatus 100 performs step S251. At step S251, the execution device 51 of the CPAP apparatus 100 transmits the update date-and-time data DTU to the server 200. The execution device 51 of the CPAP apparatus 100 also transmits identification information for identifying the CPAP apparatus 100 to the server 200. In the present embodiment, step S251 is an example of an update time acquisition process. When the server 200 receives the update date-and-time data DTU, the execution device 210 of the server 200 proceeds to step S253.

At step S253, the execution device 210 of the server 200 obtains the expiration date-and-time data DE stored in the storage device 220. This step is an example of an expiration date-and-time acquisition process that is performed, when an update of the client-side configuration data DS1 including the basic settings DSB is requested, to obtain the expiration date-and-time data DE indicating the expiration date and time of the server-side configuration data DS2 to be transmitted for the update. The execution device 210 of the server 200 then determines whether the current date and time is on or before the expiration date and time indicated by the expiration date-and-time data DE. In other words, the current date and time is the date and time when the update of the client-side configuration data DS1 including the basic settings DSB is requested.

When the execution device 210 determines at step S253 that the current date and time is after the expiration date and time indicated by the expiration date-and-time data DE (S253: NO), the execution device 210 proceeds to step S255.

At step S255, the execution device 210 of the server 200 performs a warning process for warning the user of the CPAP apparatus 100. For example, the execution device 210 of the server 200 outputs a control signal to the CPAP device 100 to warn the user of the CPAP apparatus 100, via the display 30 of the CPAP device 100, that the current date and time is past the expiration date and time of the server-side configuration data DS2. In this case, the execution device 210 of the server 200 terminates the current basic setting update control.

On the other hand, when the execution device 210 determines at step S253 that the current date and time is on or before the expiration date and time indicated by the expiration date-and-time data DE (S253: YES), the execution device 210 proceeds to step S254. In other words, the execution device 210 performs one of a first update process and a second update process described below on condition that the current date and time is on or before the expiration date and time indicated by the expiration date-and-time data DE.

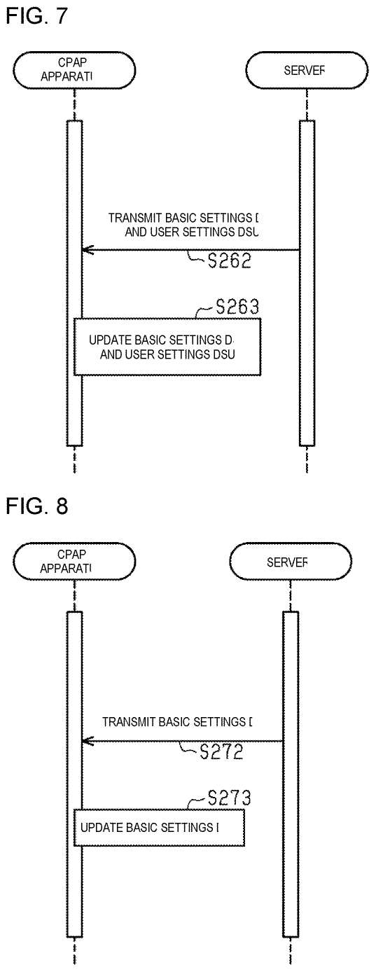

At step S254, the execution device 210 of the server 200 obtains the generation date-and-time data DTC stored in the storage device 220. This step is performed, when an update of the client-side configuration data DS1 including the basic settings DSB is requested, to obtain the generation date-and-time data DTC indicating the generation date and time of the server-side configuration data DS2 to be transmitted for the update. In other words, this step is an example of a generation time acquisition process. The execution device 210 of the server 200 then determines whether the update date and time indicated by the update date-and-time data DTU is after the generation date and time indicated by the generation date-and-time data DTC. When the execution device 210 determines at step S254 that the update date and time indicated by the update date-and-time data DTU is on or before the generation date and time indicated by the generation date-and-time data DTC, the execution device 210 proceeds to step S262 in FIG. 7. In other words, the execution device 210 proceeds to step S262 when the update date and time indicated by the update date-and-time data DTU is on or before the generation date and time indicated by the generation date-and-time data DTC.

As illustrated in FIG. 7, at step S262, the execution device 210 of the server 200 transmits the basic settings DSB and the user settings DSU in the server-side configuration data DS2 associated with the generation date-and-time data DTC to the CPAP apparatus 100. At this step, the execution device 210 of the server 200 transmits the basic settings DSB and so on of the CPAP apparatus 100 associated with the identification information obtained at step S251 to the CPAP apparatus 100 associated with the identification information obtained at step S251. When the CPAP apparatus 100 receives the server-side configuration data DS2, the execution device 51 of the CPAP apparatus 100 proceeds to step S263.

At step S263, the execution device 51 of the CPAP apparatus 100 updates the basic settings DSB and the user settings DSU in the client-side configuration data DS1 with the basic settings DSB and the user settings DSU in the server-side configuration data DS2 associated with the generation date-and-time data DTC. In the present embodiment, a process including steps S262 and S263 is an example of a first update process in which the execution device 210 of the server 200 transmits the basic settings DSB and the user settings DSU in the server-side configuration data DS2 associated with the generation date-and-time data DTC to the CPAP apparatus 100 to update the basic settings DSB and the user settings DSU in the client-side configuration data DS1 with the basic settings DSB and the user settings DSU in the server-side configuration data DS2 associated with the generation date-and-time data DTC. After step S263, the execution device 51 terminates the current basic setting update control.

On the other hand, when the execution device 210 determines at step S254 that the update date and time indicated by the update date-and-time data DTU is after the generation date and time indicated by the generation date-and-time data DTC, the execution device 210 proceeds to step S272 in FIG. 8. In other words, the execution device 210 proceeds to step S272 when the update date and time indicated by the update date-and-time data DTU is after the generation date and time indicated by the generation date-and-time data DTC.

As illustrated in FIG. 8, at step S272, the execution device 210 of the server 200 transmits the basic settings DSB in the server-side configuration data DS2 associated with the generation date-and-time data DTC to the CPAP apparatus 100. At this step, the execution device 210 of the server 200 transmits the basic settings DSB of the CPAP apparatus 100 associated with the identification information obtained at step S251 to the CPAP apparatus 100 associated with the identification information obtained at step S251. When the CPAP apparatus 100 receives the server-side configuration data DS2, the execution device 51 of the CPAP apparatus 100 proceeds to step S273.

At step S273, the execution device 51 of the CPAP apparatus 100 updates the basic settings DSB in the client-side configuration data DS1 with the basic settings DSB in the server-side configuration data DS2 associated with the generation date-and-time data DTC. In this case, the execution device 51 of the CPAP apparatus 100 keeps the user settings DSU in the client-side configuration data DS1 unchanged. In the present embodiment, a process including steps S272 and S273 is an example of a second update process in which the execution device 210 of the server 200 transmits the basic settings DSB in the server-side configuration data DS2 associated with the generation date-and-time data DTC to the CPAP apparatus 100 to update the basic settings DSB in the client-side configuration data DS1 with the basic settings DSB in the server-side configuration data DS2 associated the generation date-and-time data DTC, while keeping the user settings DSU in the client-side configuration data DS1 unchanged. After step S273, the execution device 51 terminates the current basic setting update control.

### (Effects of Present Embodiment)

The present embodiment achieves the same effects as those described above in (1-1) to (1-3).

### (Variations)

The first embodiment and the second embodiment may be modified as described below. The first embodiment, the second embodiment, and variations described below may be combined with each other as long as they do not technically conflict with each other.
In the first embodiment, the basic setting update control may be modified. For example, the warning process at step S155 may be omitted. More specifically, when there is little need to warn the user of the CPAP apparatus 100 that the expiration date and time of the server-side configuration data DS2 has passed, the warning process of step S155 may be omitted. In this case, when the execution device 51 determines at step S153 that the current date and time is after the expiration date and time indicated by the expiration date-and-time data DE, the execution device 51 may simply terminate the current basic setting update control without performing the warning process of step S155. This modification may also be applied to the second embodiment.
For example, step S153 may be omitted. More specifically, when it is only necessary to prevent an unintentional update of the user settings DSU set by the user, the execution device 51 may be configured to not perform the process of comparing the current date and time with the expiration date and time indicated by the expiration date-and-time data DE at step S153. With this configuration, the execution device 51 does not have to perform the expiration date-and-time acquisition process to obtain the expiration date-and-time data DE. Also, the storage device 220 of the server 200 does not have to store the expiration date-and-time data DE. This modification may also be applied to the second embodiment.
In the first embodiment described above, the update system SU may also perform other processes. For example, the execution device 51 of the CPAP apparatus 100 may perform a schedule reception process for receiving a scheduled date and time when the user of the CPAP apparatus 100 is to update the client-side configuration data DS1 including the basic settings DSB. Furthermore, when a scheduled date and time is received in the schedule reception process, the execution device 51 of the CPAP apparatus 100 may perform an expiration date-and-time acquisition process for obtaining the expiration date-and-time data DE indicating the expiration date and time of the server-side configuration data DS2 to be obtained to perform the update at the scheduled date and time. Then, the execution device 51 of the CPAP apparatus 100 may perform one of the first update process and the second update process on condition that the scheduled date and time is on or before the expiration date and time indicated by the expiration date-and-time data DE and the current date and time is on or after the scheduled date and time. This configuration makes it possible to prevent the first update process and the second update process from being performed to update the client-side configuration data DS1 with the expired server-side configuration data DS2 at the scheduled date and time received in the schedule reception process. This modification may also be applied to the second embodiment.
For example, when the execution device 51 of the CPAP apparatus 100 is configured to perform the schedule reception process and the expiration date-and-time acquisition process described above, the execution device 51 of the CPAP apparatus 100 may perform the warning process for warning the user of the CPAP apparatus 100 when the scheduled date and time is after the expiration date and time indicated by the expiration date-and-time data DE. More specifically, the execution device 51 may output a control signal to the display 30 to warn the user of the CPAP apparatus 100, via the display 30, that the scheduled date and time is after the expiration date and time of the server-side configuration data DS2. This configuration enables the user of the CPAP apparatus 100 to know that the scheduled date and time is after the expiration date and time of the server-side configuration data DS2. This modification may also be applied to the second embodiment.
As a setting of a scheduled time, the execution device 51 does not have to receive a date and time. For example, in the schedule reception process, the execution device 51 may set a scheduled time by receiving a time period, such as 10 days, from the time when the schedule reception process is performed.
In the first embodiment, the configuration of the update system SU may be modified. For example, update time data indicating the update time of the user settings DSU may be modified. That is, update time data is not limited to the update date-and-time data DTU. More specifically, update time data may be data indicating an elapsed time period from a predetermined reference date and time. Even in this case, it is possible to identify the update date and time of the user settings DSU based on the predetermined reference date and time and the elapsed time period indicated by the update time data. This modification may also be applied to the second embodiment.
For example, generation time data indicating the generation time of the server-side configuration data DS2 may be modified. That is, generation time data is not limited to the generation date-and-time data DTC. More specifically, generation time data may indicate an elapsed time period from a predetermined reference date and time. Even in this case, it is possible to identify the generation date and time of the server-side configuration data DS2 based on the predetermined reference date and time and the elapsed time period indicated by the generation time data. Here, when the reference date and time used for the generation time data is the same as the reference date and time used for the update time data, it is not necessary to identify the generation date and time of the server-side configuration data DS2 and the update date and time of the user settings DSU. That is, at step S154, the execution device 51 of the CPAP apparatus 100 may compare the elapsed time period indicated by the generation time data with the elapsed time period indicated by the update time data. This modification may also be applied to the second embodiment.
For example, data indicating the expiration date and time of the server-side configuration data DE may be modified. Specifically, the expiration date-and-time data DE may indicate the elapsed time period from a predetermined reference date and time. Here, an example of a predetermined reference date and time is the generation date and time indicated by the generation date-and-time data DTC. This modification may also be applied to the second embodiment.
For example, the body 10 of the CPAP apparatus 100 does not have to include the operation switches 20. As a specific example, assume that the control device 50 of the CPAP apparatus 100 can communicate with an input device outside the CPAP apparatus 100 by, for example, wireless communication. With this configuration, the control device 50 can receive operations performed by the user of the CPAP apparatus 100 using the input device even when the control device 50 does not include the operation switches 20. An example of the input device described above is a so-called smartphone. Also, an example of a communication method for the wireless communication described above is Bluetooth (registered trademark).
For example, the body 10 of the CPAP apparatus 100 does not have to include the display 30. As an example, assume that the control device 50 of the CPAP apparatus 100 can communicate with a display device outside the CPAP apparatus 100 by, for example, wireless communication. With this configuration, the control device 50 can provide various types of information to the user of the CPAP apparatus 100 via the display device even when the control device 50 does not include the display 30. An example of the display device is a so-called smartphone. An example of a communication method for the wireless communication described above is wireless communication conforming to a Wi-Fi standard.
For example, when it is only necessary to prevent an unintentional update of the user settings DSU set by the user, the CPAP apparatus 100 does not have to include the hose 80 and the mask 90.
For example, the update system SU may include a portable storage medium instead of the server 200. In other words, the execution device 51 of the CPAP apparatus 100 may obtain configuration data DS and so on stored in a portable storage medium when updating the client-side configuration data DS1 including the basic settings DSB. In this configuration, the portable storage medium may store the configuration data DS, the generation date-and-time data DTC, and the expiration date-and-time data DE. An example of the portable storage medium is a so-called SD memory card. An SD memory card may also be referred to as an SD card.

### (Other Technical Ideas)

Technical ideas that can be obtained from the above embodiments and variations are described below.
[1] In the above-described CPAP apparatus, the execution device may perform a schedule reception process for receiving a scheduled update date and time when an update of the configuration data including the basic settings is to be performed by the specific user, an expiration date-and-time acquisition process that is performed when the scheduled update date and time is received in the schedule reception process to obtain expiration date-and-time data indicating the expiration date and time of the configuration data to be obtained to perform the update at the scheduled update date and time, and one of the first update process and the second update process on condition that the scheduled update date and time is on or before the expiration date and time indicated by the expiration date-and-time data and the current date and time is on or after the scheduled update date and time.
[2] In the CPAP apparatus as described in [1] above, the execution device may perform a warning process for warning the specific user when the scheduled date and time is after the expiration date and time indicated by the expiration date-and-time data.

What is claimed is:

1. A CPAP apparatus comprising:
a storage device that stores configuration data of the CPAP apparatus; and
an execution device,
wherein the configuration data includes a modifiable user setting and a basic setting that is not modifiable by a specific user, and
wherein the execution device is configured to:
update the user setting in the configuration data in response to a request by the specific user to update the user setting,
store an update time of the user setting as update time data in the storage device when the user setting is updated,
obtain, when an update of the configuration data including the basic setting is requested, generation time data indicating a generation time of the configuration data to be obtained to perform the update,
update the basic setting and the user setting in the configuration data stored in the storage device with the basic setting and the user setting in the configuration data associated with the generation time data when the update time indicated by the update time data stored in the storage device is on or before the generation time indicated by the generation time data, and
update the basic setting in the configuration data stored in the storage device with the basic setting in the configuration data associated with the generation time data while keeping the user setting in the configuration data stored in the storage device unchanged when the update time indicated by the update time data stored in the storage device is after the generation time indicated by the generation time data.

2. The CPAP apparatus according to claim 1,
wherein when the update of the configuration data including the basic setting is requested, the execution device is configured to obtain expiration date-and-time data indicating an expiration date and time of the configuration data to be obtained to perform the update; and
wherein the execution device is further configured to update the basic setting and the user setting in the configuration data stored in the storage on condition that a current date and time is on or before the expiration date and time indicated by the expiration date-and-time data.

3. The CPAP apparatus according to claim 2, wherein the execution device is further configured to warm the specific user when the current date and time is after the expiration date and time indicated by the expiration date-and-time data.

4. A CPAP configuration data update system comprising:
a CPAP apparatus comprising a client-side storage device that stores configuration data of the CPAP apparatus and a client-side execution device; and
a server comprising a server-side storage device that stores the configuration data and a server-side execution device that is configured to update the configuration data of the CPAP apparatus,
wherein the configuration data includes a modifiable user setting and a basic setting that is not modifiable by a specific user;
wherein the client-side execution device is configured to:
update the user setting in the configuration data in response to a request by the specific user to update the user setting, and
store an update time of the user setting as update time data in the client-side storage device when the user setting is updated; and
wherein the server-side execution device is configured to:
obtain generation time data indicating a generation time of the configuration data stored in the server-side storage device when an update of the configuration data including the basic setting at the CPAP apparatus is requested,
obtain the update time data stored in the client-side storage device when the update of the configuration data including the basic setting at the CPAP apparatus is requested,
update the basic setting and the user setting in the configuration data stored in the client-side storage device with the basic setting and the user setting in the configuration data associated with the generation time data by transmitting the basic setting and the user setting in the configuration data associated with the generation time data to the CPAP apparatus when the update time indicated by the update time data is on or before the generation time indicated by the generation time data, and
update the basic setting in the configuration data stored in the client-side storage device with the basic setting in the configuration data associated with the generation time data while keeping the user setting in the configuration data stored in the client-side storage device unchanged by transmitting the basic setting in the configuration data associated with the generation time data to the CPAP apparatus when the update time indicated by the update time data is after the generation time indicated by the generation time data.

5. A CPAP configuration data update method for a CPAP apparatus that comprises a storage device that stores configuration data of the CPAP apparatus and an execution device, the configuration data including a modifiable user setting and a basic setting that is not modifiable by a specific user, the CPAP configuration data update method being performed by the execution device and comprising:
updating the user setting in the configuration data in response to a request by the specific user to update the user setting;
storing an update time of the user setting as update time data in the storage device when the user setting is updated;
obtaining, when an update of the configuration data including the basic setting is requested, generation time data indicating a generation time of the configuration data to be obtained to perform the update;
updating the basic setting and the user setting in the configuration data stored in the storage device with the basic setting and the user setting in the configuration data associated with the generation time data when the update time indicated by the update time data stored in the storage device is on or before the generation time indicated by the generation time data; and
updating the basic setting in the configuration data stored in the storage device with the basic setting in the configuration data associated with the generation time data while keeping the user setting in the configuration data stored in the storage device unchanged when the update time indicated by the update time data stored in the storage device is after the generation time indicated by the generation time data.
